(19) United States
(12) Patent Application Publication
(10) Pub. No.: US 2026/0028458 A1
(43) Pub. Date: Jan. 29, 2026

(54) HIGH-DURABILITY GLYCOSAMINOGLYCAN GELS AND METHODS OF MAKING THE SAME

(71) Applicant: Galderma Holding SA, Zug (CH)

(72) Inventors: Lina KANDELIN, Uppsala (SE); Kristoffer BERGMAN, Sollentuna (SE)

(73) Assignee: Galderma Holding SA, Zug (CH)

(21) Appl. No.: 19/278,582

(22) Filed: Jul. 23, 2025

Related U.S. Application Data
(60) Provisional application No. 63/674,969, filed on Jul. 24, 2024.

(51) Int. Cl. C08J 3/075; A61L 27/20; C08J 3/24; C08K 5/17

(52) U.S. Cl. CPC C08J 3/075; A61L 27/20; C08J 3/24; C08K 5/17; A61L 2400/06; C08J 2305/08

(57) ABSTRACT

Disclosed are durable polysaccharide compositions comprising GAGs covalently crosslinked with a carbohydrate crosslinker, and methods of making the same. GAG hydrogels according to the present disclosure have a molecular weight of at least 1.5 MDa, crosslinked by a crosslinker forming amide bonds with the GAG backbone and an elastic modulus (G′) of less than or equal to 200 Pa. Further described are methods of using the durable polysaccharide compositions for treating skin (e.g., in reparative or plastic surgery, esthetic dermatology, facial contouring, body contouring, or gingival augmentation).

### CROSS-REFERENCE TO RELATED APPLICATIONS

This application claims priority to U.S. Patent Application No. 63/674,969, filed Jul. 24, 2024, the entire contents of which are hereby incorporated by reference herein.

### FIELD

The present disclosure concerns formulations of high-molecular-weight (“high Mw”) glycosaminoglycans (“GAGs”) (e.g., hyaluronic acid (“HA”)) for medical and/or cosmetic applications. In particular, the present disclosure relates to hydrogels containing crosslinked GAGs for use in medical and/or cosmetic applications such as implants for subcutaneous or intradermal injection, which may be used in reparative or plastic surgery and in esthetic dermatology. The crosslinked GAGs are particularly stable compared to conventional gels.

### BACKGROUND

The following description of the background of the present technology is provided simply as an aid in understanding the present technology and is not admitted to describe or constitute prior art to the present technology.

Hydrogels are often prepared by chemically crosslinking polymers to form large polymeric networks. While both monomeric and minimally polymerized polysaccharides absorb water to the point of saturation, polysaccharides dissolve at the point of saturation, while hydrogels comprising the same polysaccharides-albeit in a crosslinked form-typically can absorb water without dissolving, resulting in swelling. Glycosaminoglycans (“GAGs”) are negatively-charged, long, linear polysaccharides with a capacity to absorb large amounts of water. Biocompatible GAGs commonly used hydrogels for medical and cosmetic applications include hyaluronic acid (“HA”), chondroitin, and chondroitin sulfate. Of these, HA and its derivatives are among the most widely used biocompatible polymers in medicine. However, HA has low durability in vivo, so chemical modification via crosslinking or other means is required to improve the in vivo durability of HA.

The present disclosure overcomes problems associated with preparing hydrogels from high-Mw GAGs by enhancing durability, enabling the hydrogels to remain stable during degradation conditions (e.g., heat sterilization) while maintaining the ability to dilute hydrogels to desired GAG concentrations for medical applications, such as filling syringes with hydrogels.

### SUMMARY

The present disclosure concerns methods of producing hydrogels comprising crosslinked high-Mw GAGs capable of maintaining their structural integrity under conditions that would otherwise initiate hydrolysis or phase separation. The present disclosure is further drawn to hydrogel products thus obtained, along with methods of cosmetically treating skin using the same.

In one aspect, which may be combined with any other aspect or embodiment, the present disclosure relates to a method of preparing a hydrogel comprising crosslinked glycosaminoglycan (GAG) molecules, the method comprising: crosslinking a GAG having a molecular weight of at least 1.5 MDa with a crosslinker to obtain a GAG hydrogel crosslinked by amide bonds, wherein: the concentration of GAG is less than 2 wt. % during the crosslinking; the molar ratio of the crosslinker to the GAG is greater than or equal to 1.5 mol % per GAG disaccharide during the crosslinking; and the GAG hydrogel has an elastic modulus (G′) of less than or equal to 200 Pa.

In some embodiments, the GAG comprises hyaluronic acid (HA), chondroitin, or chondroitin sulfate. In some embodiments, the GAG comprises HA.

In some embodiments, the crosslinker comprises a di- or multi-nucleophile functional crosslinker. In some embodiments, the crosslinker comprises an aliphatic or aromatic diamino derivative, a peptide, or a peptide sequence. In some embodiments, the crosslinker comprises one or more selected from the group consisting of di-, tri-, tetra-, and oligosaccharides. In some embodiments, the crosslinker comprises diaminotrehalose (DATH).

In some embodiments, the GAG has a weight average molecular weight of greater than or equal to about 2 MDa. In some embodiments, the GAG has a weight average molecular weight of about 2 MDa to about 10 MDa. In some embodiments, the GAG has a weight average molecular weight of greater than or equal to about 2.5 MDa. In some embodiments, the GAG has a weight average molecular weight of about 2.5 MDa to about 10 MDa. In some embodiments, the GAG has a weight average molecular weight of greater than or equal to about 3 MDa. In some embodiments, the GAG has a weight average molecular weight of about 3 MDa to about 10 MDa.

In some embodiments, during the crosslinking the GAG is present at a concentration of greater than or equal to about 1.0 wt. % and less than 2 wt. %. In some embodiments, during the crosslinking, the GAG is present at a concentration of less than or equal to about 1.8 wt. %. In some embodiments, the GAG is present at a concentration of greater than or equal to about 1.0 wt. % and less than or equal to about 1.8 wt. %. In some embodiments, during the crosslinking, the GAG is present at a concentration of less than or equal to about 1.5 wt. %. In some embodiments, during the crosslinking, the GAG is present at a concentration of greater than or equal to about 1.0 wt. % and less than or equal to about 1.5 wt. %.

In some embodiments, during the crosslinking, the molar ratio of the crosslinker to the GAG is greater than or equal to 1.5 mol % per GAG disaccharide and less than or equal to 10 mol % per GAG disaccharide. In some embodiments, during the crosslinking, the molar ratio of the crosslinker to the GAG is greater than or equal to 1.5 mol % per GAG disaccharide and less than or equal to 8 mol % per GAG disaccharide.

In some embodiments, the crosslinking comprises: crosslinking activated GAG molecules via activated carboxyl groups using a di- or multi-nucleophile functional crosslinker to obtain a GAG hydrogel crosslinked by amide bonds. In some embodiments, the crosslinking comprises: activating carboxyl groups on GAG molecules with a coupling agent to form activated GAG molecules; and crosslinking the activated GAG molecules via activated carboxyl groups using a di- or multi-nucleophile functional crosslinker to obtain a GAG hydrogel crosslinked by amide bonds.

In some embodiments, the coupling agent is a triazine-based coupling agent. In some embodiments, the coupling agent comprises 4-(4,6-dimethoxy-1,3,5-triazin-2-yl)-4-methylmorpholinium chloride (DMTMM). In some embodiments, the coupling agent comprises N-(3-dimethylanninopropyl)-N′-ethylcarbodiimide (EDC) and N-hydroxysuccinimide (NHS).

In some embodiments, the crosslinking is performed at a pH of 5 to 9. In some embodiments, the crosslinking is performed at a pH of 6 to 8.

In some embodiments, the GAG hydrogel has an elastic modulus (G′) of less than or equal to 180 Pa. In some embodiments, the GAG hydrogel has an elastic modulus (G′) of less than or equal to 150 Pa.

In some embodiments, the method further comprises sterilizing the GAG hydrogel.

In some embodiments, the GAG hydrogel product has a degradation rate of less than or equal to 2.0% per hour at 90° C. In some embodiments, the GAG hydrogel has a degradation rate of less than or equal to 1.0% per hour at 90° C.

In some embodiments, the GAG hydrogel has a GelC_{N} of greater than or equal to about 90% after 8 hr at 90° C. In some embodiments, the GAG hydrogel has a GelC_{N} of greater than or equal to about 80% after 16 hr at 90° C. In some embodiments, the GAG hydrogel has a GelC_{N} of greater than or equal to about 75% after 24 hr at 90° C. In some embodiments, the GAG hydrogel has a GelC_{N} of greater than or equal to about 80% after 24 hr at 90° C.

In another aspect, which may be combined with any other aspect or embodiment, the present disclosure relates to a GAG hydrogel comprising crosslinked glycosaminoglycan (GAG) molecules, wherein the GAG hydrogel is prepared according to a method of the present disclosure.

In another aspect, which may be combined with any other aspect or embodiment, the present disclosure relates to a GAG hydrogel, comprising: hyaluronic acid (HA) molecules crosslinked by diaminotrehalose (DATH) through amide bonds between the HA molecules and the DATH, wherein: the HA has a weight average molecular weight of greater than or equal to 2 MDa; the molar ratio of the crosslinker to the HA is greater than or equal to 1.5 mol % per GAG disaccharide; and the GAG hydrogel has an elastic modulus (G′) of less than or equal to 200 Pa.

In some embodiments, the HA has a weight average molecular weight of greater than or equal to 3 MDa; and the molar ratio of the crosslinker to the HA is 2 to 8 mol % per GAG disaccharide.

In some embodiments, the GAG hydrogel has an elastic modulus (G′) of less than or equal to 180 Pa. In some embodiments, the GAG hydrogel has an elastic modulus (G′) of less than or equal to 150 Pa.

In some embodiments, the GAG hydrogel product has a degradation rate of less than or equal to −2.0% per hour at 90° C. In some embodiments, the GAG hydrogel has a degradation rate of less than or equal to −1.0% per hour at 90° C.

In some embodiments, the GAG hydrogel has a GelC_{N} of greater than or equal to about 90% after 8 hr at 90° C. In some embodiments, the GAG hydrogel has a GelC_{N} of greater than or equal to about 80% after 16 hr at 90° C. In some embodiments, the GAG hydrogel has a GelC_{N} of greater than or equal to about 75% after 24 hr at 90° C. In some embodiments, the GAG hydrogel has a GelC_{N} of greater than or equal to about 80% after 24 hr at 90° C.

In another aspect, which may be combined with any other aspect or embodiment, the present disclosure relates to a GAG hydrogel, comprising: GAG molecules crosslinked by a crosslinker through amide bonds between the GAG molecules and the crosslinker, wherein: the GAG hydrogel has an elastic modulus (G′) of less than or equal to 200 Pa; and the GAG hydrogel has a degradation rate of less than or equal to 2.0% per hour at 90° C.

In some embodiments, the GAG has a weight average molecular weight of greater than or equal to 1.5 MDa. In some embodiments, the GAG has a weight average molecular weight of greater than or equal to 3 MDa.

In some embodiments, the molar ratio of the crosslinker to the GAG is 2 to 8 mol % per GAG disaccharide.

In some embodiments, the GAG hydrogel has an elastic modulus (G′) of less than or equal to 180 Pa. In some embodiments, the GAG hydrogel has an elastic modulus (G′) of less than or equal to 150 Pa.

In some embodiments, the GAG hydrogel product has a degradation rate of less than or equal to 2.0% per hour at 90° C. In some embodiments, the GAG hydrogel has a degradation rate of less than or equal to 1.0% per hour at 90° C. In some embodiments, the GAG hydrogel has a GelC_{N} of greater than or equal to about 90% after 8 hr at 90° C. In some embodiments, the GAG hydrogel has a GelC_{N} of greater than or equal to about 80% after 16 hr at 90° C. In some embodiments, the GAG hydrogel has a GelC_{N} of greater than or equal to about 75% after 24 hr at 90° C.

In some embodiments, the GAG is HA. In some embodiments, the crosslinker is DATH.

In another aspect, which may be combined with any other aspect or embodiment, the present disclosure relates to a hydrogel composition, comprising: the GAG hydrogel according to any of the embodiments disclosed herein; and water.

In some embodiments, the hydrogel composition further comprises a local anesthetic. In some embodiments, the local anesthetic comprises lidocaine.

In another aspect, which may be combined with any other aspect or embodiment, the present disclosure relates to a pre-filled syringe comprising a GAG hydrogel according to any of the embodiments disclosed herein.

In another aspect, which may be combined with any other aspect or embodiment, the present disclosure relates to a method of treating skin, the method comprising: administering to skin a composition comprising a GAG hydrogel according to any of the embodiments disclosed herein.

In another aspect, which may be combined with any other aspect or embodiment, the present disclosure relates to a method of performing a reparative or esthetic dermatologic treatment, the method comprising: administering to skin a composition comprising a GAG hydrogel according to any of the embodiments disclosed herein.

In some embodiments, the administering comprises an injection of the composition comprising the GAG hydrogel. In some embodiments, the injection is a subdermal, intradermal, subcutaneous, intramuscular, submuscular, or intragingival injection.

In another aspect, which may be combined with any other aspect or embodiment, the present disclosure relates to a method of performing dermal filling, the method comprising: injecting a subject with a composition comprising a GAG hydrogel according to any of the embodiments disclosed herein. In some embodiments, the injecting is performed to fill fine lines or fine wrinkles in the subject's face, neck, hands, feet, knees, or elbows. In some embodiments, the injecting is performed to fill scars. In some embodiments, the scars comprise depressed scars, hypertrophic scars, keloid scars, or a combination thereof.

In another aspect, which may be combined with any other aspect or embodiment, the present disclosure relates to a method of performing facial contouring, the method comprising: injecting a subject's face with a composition comprising a GAG hydrogel according to any of the embodiments disclosed herein.

In another aspect, which may be combined with any other aspect or embodiment, the present disclosure relates to a method of performing body contouring, the method comprising: injecting a subject with a composition comprising a GAG hydrogel according to any of the embodiments disclosed herein. In some embodiments, the injecting modifies the size and shape of the breasts, buttocks, sacrum, groin, hips, abdomen, thorax, feet, legs, knees, popliteus, thighs, arms, hands, elbows, antecubitis, or a combination thereof.

Additional aspects and/or embodiments of the invention will be provided, without limitation, in the detailed description of the present technology set forth below. The following detailed description is exemplary and explanatory, but it is not intended to be limiting.

### BRIEF DESCRIPTION OF THE DRAWINGS

Various objects, aspects, features, and advantages of the disclosure will become more apparent and better understood by referring to the detailed description taken in conjunction with the accompanying figures.

FIG. 1 is a plot of normalized GelC (%) versus time for gels incubated at 90° C.

FIG. 2 is a plot of normalized GelC (%) versus time for gels incubated at 90° C.

### DETAILED DESCRIPTION

### Hydrogels and Methods of Making Hydrogels

Fillers such as dermal fillers have been used to repair, restore or augment hard or soft tissue contour defects of the body due to aging, injury, or acquired or congenital deformities of the face, body and internal organs. Fillers may be natural or synthetic substances that are used to reduce wrinkles and/or fine lines, restore lost volume, hydrate the skin, soften nasolabial folds, augment and contour lips, improve scars (depressed, hypertrophic and keloid scars), strengthen weakened vocal cords, and provide other soft tissue improvements. Substances that have been utilized include fat, paraffin, human collagen, bovine collagen, silicone, hyaluronic acids, lactic acids, and glycolic acids. In 1981, a new era in soft tissue fillers emerged with the FDA approval of bovine collagen. Since then, many soft tissue fillers have emerged. The dramatic increase in the number of current and investigational fillers has been fueled by many factors including improvements in biotechnology and an emphasis on cosmetic appearance in society. With the introduction of newer fillers, there has been an ongoing need to evaluate their risk/benefit profiles and define their limitations in order to maximize patient cosmetic outcomes and safety. Common filler/hydrogel compositions include GAGs such as hyaluronic acid.

### Methods of Producing GAG Hydrogels

The present disclosure relates to a method of preparing a hydrogel comprising crosslinked glycosaminoglycan (GAG) molecules, the method comprising: crosslinking a GAG having a molecular weight of at least 1.5 MDa with a crosslinker to obtain a GAG hydrogel crosslinked by amide bonds, wherein: the concentration of GAG is less than 2 wt. % during the crosslinking; the molar ratio of the crosslinker to the GAG is greater than or equal to 1.5 mol % per GAG disaccharide during the crosslinking; and the GAG hydrogel has an elastic modulus (G′) of less than or equal to 200 Pa.

Methods of producing GAG hydrogels are disclosed in PCT publication numbers WO2017/114867, WO2017/114861, WO2017/114864, and WO2017/114865; US Patent Publication Numbers US20190023812A1, US20190016830A1, US20190023855A1, and US20070066816A1; and U.S. Pat. Nos. 8,858,999, 6,831,172, 8,887,243, and, 6,703,444.

In some embodiments, methods for producing GAG hydrogels comprise: at least partially deacetylating a GAG comprising acetyl groups, the deacetylating comprising: (a) providing a GAG comprising acetyl groups; (b) reacting the GAG comprising acetyl groups with hydroxylamine or a salt thereof at a temperature of 100° C. or less for 2 to 200 hours to form an at least partially deacetylated GAG, and (c) recovering the at least partially deacetylated GAG. Such methods are described in, e.g., WO 2017/114861. In some embodiments, an at least partially deacetylated GAG is provided as a GAG for crosslinking according to the present disclosure.

In some embodiments, methods for producing GAG hydrogels comprise: (a) providing a GAG crosslinked by amide bonds, wherein the crosslinked GAGs comprise residual amine groups; and (b) acylating residual amine groups of the crosslinked GAGS provided in (a) to form acylated crosslinked GAGs. Such methods are described in, e.g., WO 2017/114864. In some embodiments, GAG hydrogels produced according to the present disclosure may be subjected to acylation of residual amine groups.

In some embodiments, methods for producing GAG hydrogels comprise: (a) providing a GAG crosslinked by amide bonds, wherein the crosslinked GAG comprises ester crosslinks formed as byproducts during amide crosslinking; and (b) subjecting the crosslinked GAGs to alkaline treatment to hydrolyze ester crosslinks formed as byproducts during the amide crosslinking. Such methods are described in, e.g., WO 2017/114865. In some embodiments, GAG hydrogels produced according to the present disclosure may be subjected to alkaline treatment to hydrolyze ester crosslinks.

In some embodiments, methods for producing GAG hydrogels comprise: (a) crosslinking activated carboxyl groups of HA molecules using diaminotrehalose (DATH) to obtain crosslinked HA molecules; and (b) hydrolyzing ester bonds in the crosslinked HA molecules via alkaline hydrolysis to obtain a hydrogel product having less than 50% non-crosslinked HA molecules by weight of the hydrogel product. Such methods are described, e.g., in WO 2017/114867.

### Glycosaminoglycans (GAGs)

Hydrogel products according to the present disclosure comprise crosslinked glycosaminoglycan (GAG) molecules. GAGs are negatively-charged, long, linear heteropolysaccharides with a capacity to absorb large amounts of water. Biocompatible GAGs commonly used in medical and cosmetic applications include hyaluronic acid (“HA”), chondroitin, and chondroitin sulfate. Of these, HA and its derivatives are among the most widely used biocompatible polymers for medical use.

In some embodiments, the GAG comprises or consists of a sulfated or non-sulfated GAG such as hyaluronic acid, chondroitin, chondroitin sulfate, heparan sulfate, heparosan, heparin, dermatan sulfate, keratan sulfate, or a combination thereof. In some embodiments, the GAG comprises hyaluronic acid, chondroitin, chondroitin sulfate, or a combination thereof. In some embodiments, the GAG comprises hyaluronic acid. In some embodiments, the GAG molecules comprise one or more types of GAGs.

In some embodiments, the GAG is a native GAG. In some embodiments, the GAG is used in its native state (i.e., the chemical structure of the GAG has not been altered or modified by addition of functional groups or other chemical moieties). Using the GAG in its native state affords a crosslinked structure more closely resembling the natural molecules, which conserves the native properties and effects of the GAG itself and can minimize the immune response when the crosslinked GAG is introduced into the body. In some embodiments, the GAG is a naturally-occurring GAG.

As used herein, the terms “hyaluronic acid” or “HA” encompass all variants and combinations of variants of hyaluronic acid, hyaluronate, or hyaluronan—of various chain lengths and charge states, as well as various chemical modifications, including crosslinking. In some embodiments, “hyaluronic acid” or “HA” encompass the various hyaluronate salts of hyaluronic acid with various counter ions (e.g., sodium hyaluronate). In some embodiments, “hyaluronic acid” or “HA” encompasses modified variants of hyaluronic acid, such as oxidized variants (e.g., wherein —CH₂OH groups have been oxidized to —CHO and/or —COOH; or wherein vicinal hydroxyl groups have been oxidized using periodate oxidation; or variants in which oxidized groups (e.g., —CHO and/or —COOH) have been reduced to —CH2OH or coupled with amines to form imines followed by reduction to secondary amines; or variants modified by sulphation; or variants modified by deamidation, which may be followed by deamination or amide formation with new acids; or variants modified via esterification; crosslinked variants; substituted variants (e.g., variants modified via a crosslinking agent or a carbodiimide-assisted coupling); variants coupled to different molecules (e.g., proteins, peptides and active drug components); and deacetylated variants. In some embodiments, hyaluronic acid may be further modified by isourea, hydrazide, bromocyan, monoepoxide, and monosulfone couplings.

In some embodiments, hyaluronic acid may be obtained from various sources of animal and non-animal origin. In some embodiments, sources of non-animal origin include yeast or bacteria.

The GAG may have any suitable molecular weight (Mw) for forming a durable crosslinked hydrogel product with properties suitable for administration to a subject (e.g., for injection as a dermal filler). In some embodiments, the GAG has a Mw of greater than or equal to 0.1 MDa, greater than or equal to 0.2 MDa, greater than or equal to 0.3 MDa, greater than or equal to 0.4 MDa, greater than or equal to 0.5 MDa, greater than or equal to 0.6 MDa, greater than or equal to 0.7 MDa, greater than or equal to 0.8 MDa, greater than or equal to 0.9 MDa, greater than or equal to 1.0 MDa, greater than or equal to 1.1 MDa, greater than or equal to 1.2 MDa, greater than or equal to 1.3 MDa, greater than or equal to 1.4 MDa, greater than or equal to 1.5 MDa, greater than or equal to 1.6 MDa, greater than or equal to 1.7 MDa, greater than or equal to 1.8 MDa, greater than or equal to 1.9 MDa, greater than or equal to 2 MDa, greater than or equal to 2.1 MDa, greater than or equal to 2.2 MDa, greater than or equal to 2.3 MDa, greater than or equal to 2.4 MDa, greater than or equal to 2.5 MDa, greater than or equal to 2.6 MDa, greater than or equal to 2.7 MDa, greater than or equal to 2.8 MDa, greater than or equal to 2.9 MDa, greater than or equal to 3 MDa, greater than or equal to 3.1 MDa, greater than or equal to 3.2 MDa, greater than or equal to 3.3 MDa, greater than or equal to 3.4 MDa, greater than or equal to 3.5 MDa, greater than or equal to 4 MDa, greater than or equal to 4.5 MDa, greater than or equal to 5 MDa, greater than or equal to 5.5 MDa, greater than or equal to 6 MDa, greater than or equal to 6.5 MDa, greater than or equal to 7 MDa, greater than or equal to 7.5 MDa, greater than or equal to 8 MDa, greater than or equal to 8.5 MDa, greater than or equal to 9 MDa, greater than or equal to 9.5 MDa, greater than or equal to 10 MDa, or any range or value therein between.

In some embodiments, the GAG has a Mw of less than or equal to 20 MDa, less than or equal to 15 MDa, less than or equal to 10 MDa, less than or equal to 9.5 MDa, less than or equal to 9 MDa, less than or equal to 8.5 MDa, less than or equal to 8 MDa, less than or equal to 7.5 MDa, less than or equal to 7 MDa, less than or equal to 6.5 MDa, less than or equal to 6 MDa, less than or equal to 5.5 MDa, less than or equal to 5 MDa, less than or equal to 4.5 MDa, less than or equal to 4 MDa, less than or equal to 3.5 MDa, less than or equal to 3 MDa, less than or equal to 2.5 MDa, less than or equal to 2.4 MDa, less than or equal to 2.3 MDa, less than or equal to 2.2 MDa, less than or equal to 2.1 MDa, less than or equal to 2.0 MDa, less than or equal to 1.9 MDa, less than or equal to 1.8 MDa, less than or equal to 1.7 MDa, less than or equal to 1.6 MDa, less than or equal to 1.5 MDa, less than or equal to 1.4 MDa, less than or equal to 1.3 MDa, less than or equal to 1.2 MDa, less than or equal to 1.1 MDa, less than or equal to 1.0 MDa, less than or equal to 0.9 MDa, less than or equal to 0.8 MDa, less than or equal to 0.7 MDa, less than or equal to 0.6 MDa, less than or equal to 0.5 MDa, less than or equal to 0.4 MDa, less than or equal to 0.3 MDa, less than or equal to 0.2 MDa, less than or equal to 0.1 MDa, or any range or value therein between.

In some embodiments, the GAG has a Mw of 0.1 MDa to 20 MDa, 0.5 MDa to 20 MDa, 1 MDa to 20 MDa, 1 MDa to 15 MDa, 1 MDa to 10 MDa, 1 MDa to 5 MDa, 1 MDa to 3 MDa, 2 MDa to 20 MDa, 2 MDa to 15 MDa, 2 MDa to 10 MDa, 2 MDa to 5 MDa, 2.5 MDa to 20 MDa, 2.5 MDa to 15 MDa, 2.5 MDa to 10 MDa, 2.5 MDa to 5 MDa, 3 MDa to 20 MDa, 3 MDa to 15 MDa, 3 MDa to 10 MDa, 3 MDa to 5 MDa, 3.5 MDa to 20 MDa, 3.5 MDa to 15 MDa, 3.5 MDa to 10 MDa, 3.5 MDa to 5 MDa, 4 MDa to 20 MDa, 4 MDa to 10 MDa, 4 MDa to 5 MDa, 5 MDa to 20 MDa, 5 MDa to 10 MDa, or any range or value therein.

During crosslinking, the GAG may be present at any suitable concentration to achieve efficient crosslinking of the GAG molecules to produce durable GAG hydrogels. In some embodiments, during the crosslinking, the GAG is present at a concentration of less than or equal to about 5 wt. %, less than or equal to about 4.5 wt. %, less than or equal to about 4 wt. %, less than or equal to about 3.5 wt. %, less than or equal to about 3.4 wt. %, less than or equal to about 3.3. wt. %, less than or equal to about 3.2 wt. %, less than or equal to about 3.1 wt. %, less than or equal to about 3 wt. %, less than or equal to about 2.9 wt. %, less than or equal to about 2.8 wt. %, less than or equal to about 2.7 wt. %, less than or equal to about 2.6 wt. %, less than or equal to about 2.5 wt. %, less than or equal to about 2.4 wt. %, less than or equal to about 2.3 wt. %, less than or equal to about 2.2 wt. %, less than or equal to about 2.1 wt. %, less than or equal to about 2.0 wt. %, less than or equal to about 1.9 wt. %, less than or equal to about 1.8 wt. %, less than or equal to about 1.7 wt. %, less than or equal to about 1.6 wt. %, less than or equal to about 1.5 wt. %, less than or equal to about 1.4 wt. %, less than or equal to about 1.3 wt. %, less than or equal to about 1.2 wt. %, less than or equal to about 1.1 wt. %, less than or equal to about 1.0 wt. %, less than or equal to about 0.9 wt. %, less than or equal to about 0.8 wt. %, less than or equal to about 0.7 wt. %, less than or equal to about 0.6 wt. %, less than or equal to about 0.5 wt. %, less than or equal to about 0.4 wt. %, less than or equal to about 0.3 wt. %, less than or equal to about 0.2 wt. %, less than or equal to about 0.1 wt. %, or any range or value therein between.

In some embodiments, during the crosslinking, the GAG is present at a concentration of greater than or equal to about 0.001 wt. %, greater than or equal to about 0.005 wt. %, greater than or equal to about 0.01 wt. %, greater than or equal to about 0.05 wt. %, greater than or equal to about 0.1 wt. %, greater than or equal to about 0.2 wt. %, greater than or equal to about 0.3 wt. %, greater than or equal to about 0.4 wt. %, greater than or equal to about 0.5 wt. %, greater than or equal to about 0.6 wt. %, greater than or equal to about 0.7 wt. %, greater than or equal to about 0.8 wt. %, greater than or equal to about 0.9 wt. %, greater than or equal to about 1.0 wt. %, greater than or equal to about 1.1 wt. %, greater than or equal to about 1.2 wt. %, greater than or equal to about 1.3 wt. %, greater than or equal to about 1.4 wt. %, greater than or equal to about 1.5 wt. %, greater than or equal to about 1.6 wt. %, greater than or equal to about 1.7 wt. %, greater than or equal to about 1.8 wt. %, greater than or equal to about 1.9 wt. %, greater than or equal to about 2.0 wt. %, greater than or equal to about 2.1 wt. %, greater than or equal to about 2.2 wt. %, greater than or equal to about 2.3 wt. %, greater than or equal to about 2.4 wt. %, greater than or equal to about 2.5 wt. %, greater than or equal to about 2.6 wt. %, greater than or equal to about 2.7 wt. %, greater than or equal to about 2.8 wt. %, greater than or equal to about 2.9 wt. %, greater than or equal to about 3.0 wt. % or any range or value therein between.

In some embodiments, during crosslinking, the GAG is present at a concentration of greater than or equal to 0.001 wt. % and less than 2.0 wt. %, greater than or equal to 0.005 wt. % and less than 2.0 wt. %, greater than or equal to 0.01 wt. % and less than 2.0 wt. %, greater than or equal to 0.05 wt. % and less than 2.0 wt. %, greater than or equal to 0.1 wt. % and less than 2.0 wt. %, greater than or equal to 0.2 wt. % and less than 2.0 wt. %, greater than or equal to 0.3 wt. % and less than 2.0 wt. %, greater than or equal to 0.4 wt. % and less than 2.0 wt. %, greater than or equal to 0.5 wt. % and less than 2.0 wt. %, greater than or equal to 0.6 wt. % and less than 2.0 wt. %, greater than or equal to 0.7 wt. % and less than 2.0 wt. %, greater than or equal to 0.8 wt. % and less than 2.0 wt. %, greater than or equal to 0.9 wt. % and less than 2.0 wt. %, greater than or equal to 1.0 wt. % and less than 2.0 wt. %, greater than or equal to 1.1 wt. % and less than 2.0 wt. %, greater than or equal to 1.2 wt. % and less than 2.0 wt. %, greater than or equal to 1.3 wt. % and less than 2.0 wt. %, greater than or equal to 1.4 wt. % and less than 2.0 wt. %, greater than or equal to 1.5 wt. % and less than 2.0 wt. %.

In some embodiments, during crosslinking, the GAG is present at a concentration of 0.001 wt. % to 5.0 wt. %, 0.005 wt. % to 5.0 wt. %, 0.01 wt. % to 5.0 wt. %, 0.05 wt. % to 5.0 wt. %, 0.05 wt. % to 4.5 wt. %, 0.05 wt. % to 4.0 wt. %, 0.05 wt. % to 3.5 wt. %, 0.05 wt. % to 3.0 wt. %, 0.05 wt. % to 2.5 wt. %, 0.05 wt. % to 2.0 wt. %, 0.05 wt. % to 1.9 wt. %, 0.05 wt. % to 1.8 wt. %, 0.05 wt. % to 1.7 wt. %, 0.05 wt. % to 1.6 wt. %, 0.05 wt. % to 1.5 wt. %, 0.05 wt. % to 1.4 wt. %, 0.05 wt. % to 1.3 wt. %, 0.05 wt. % to 1.2 wt. %, 0.05 wt. % to 1.1 wt. %, 0.05 wt. % to 1.0 wt. %, 0.1 wt. % to 5.0 wt. %, 0.1 wt. % to 4.5 wt. %, 0.1 wt. % to 4.0 wt. %, 0.1 wt. % to 3.5 wt. %, 0.1 wt. % to 3.0 wt. %, 0.1 wt. % to 2.5 wt. %, 0.1 wt. % to 2.0 wt. %, 0.1 wt. % to 1.9 wt. %, 0.1 wt. % to 1.8 wt. %, 0.1 wt. % to 1.7 wt. %, 0.1 wt. % to 1.6 wt. %, 0.1 wt. % to 1.5 wt. %, 0.1 wt. % to 1.4 wt. %, 0.1 wt. % to 1.3 wt. %, 0.1 wt. % to 1.2 wt. %, 0.1 wt. % to 1.1 wt. %, 0.1 wt. % to 1.0 wt. %, 0.5 wt. % to 5.0 wt. %, 0.5 wt. % to 4.5 wt. %, 0.5 wt. % to 4.0 wt. %, 0.5 wt. % to 3.5 wt. %, 0.5 wt. % to 3.0 wt. %, 0.5 wt. % to 2.5 wt. %, 0.5 wt. % to 2.0 wt. %, 0.5 wt. % to 1.9 wt. %, 0.5 wt. % to 1.8 wt. %, 0.5 wt. % to 1.7 wt. %, 0.5 wt. % to 1.6 wt. %, 0.5 wt. % to 1.5 wt. %, 0.5 wt. % to 1.4 wt. %, 0.5 wt. % to 1.3 wt. %, 0.5 wt. % to 1.2 wt. %, 0.5 wt. % to 1.1 wt. %, 0.5 wt. % to 1.0 wt. %, 0.001 wt. % to 2 wt. %, 0.005 wt. % to 2 wt. %, 0.01 wt. % to 2 wt. %, 0.05 wt. % to 2 wt. %, 0.1 wt. % to 2 wt. %, 0.5 wt. % to 2 wt. %, or any range or value therein.

### Crosslinkers

GAGs (e.g., HA) are commonly crosslinked using a diglycidyl ether, e.g., 1,4-butanediol diglycidyl ether (BDDE) to form ester crosslinks with the GAG molecules. Alternatively, amide coupling may be performed using one or more di- or multi-amine functional crosslinkers together with a coupling agent. For example, the use of 4-(4,6-mimethoxy-1,3,5-triazin-2-yl)-4-methylmorpholinium chloride (DMTMM) for activation of carboxylic acid groups and subsequent condensation with a diamino structure (e.g., diaminotrehalose (DATH)), has proven to be an efficient method to crosslink HA with minimal degradation of the HA backbone.

The crosslinker may be any suitable crosslinker for forming stable crosslinks (e.g., intra-chain and inter-chain crosslinks) that do not quickly degrade. In some embodiments, the crosslinker comprises a di- or multi-nucleophile functional crosslinker. In some embodiments, the di- or multi-nucleophile functional crosslinker comprises two or more functional groups capable of reacting with functional carboxyl groups of the GAG, resulting in the formation of covalent bonds (e.g., amide bonds and/or ester bonds). In some embodiments, the nucleophile functional groups are capable of reacting with carboxyl groups on the glycosaminoglycan molecule to form amide bonds. In some embodiments, the nucleophile functional groups of the di-, tri-, tetra-, and oligosaccharides are selected from the group consisting of primary amine, hydrazine, hydrazide, carbazate, semi-carbazide, thiosemicarbazide, thiocarbazate and aminoxy.

In some embodiments, crosslinker is selected from the group consisting of di- or multinucleophile functional di-, tri-, tetra-, and oligo-saccharides. In some embodiments, the di- or multinucleophile functional di-, tri-, tetra-, and oligo-saccharides are derived from nucleophile functional polysaccharides, such as chitobiose derived from chitin. In some embodiments, the di- or multinucleophile functional di-, tri-, tetra-, and oligo-saccharides may also be di-, tri-, tetra-, and oligo-saccharides which have been modified by introduction of two or more nucleophile functional groups. In some embodiments, the crosslinker comprises two or more amine groups and a spacer group selected from the group consisting of di-, tri-, tetra-, and oligosaccharides. In some embodiments, the crosslinker is selected from the group consisting of diamino hyaluronic acid tetrasaccharide, diamino hyaluronic acid hexasaccharide, diamino trehalose (DATH), diamino lactose, diamino maltose, diamino sucrose, diamino chitobiose, chitobiose, or diamino raffinose. Using a carbohydrate-based crosslinker provides a hydrogel product based entirely on carbohydrate structures or derivatives thereof, which minimizes the disturbance of the crosslinking on the native properties of the GAGs utilized in producing the hydrogel.

In some embodiments, the crosslinker comprises an aliphatic or aromatic diamino derivative, a peptide, or a peptide sequence.

During the crosslinking, the crosslinker may be present at any suitable concentration to ensure efficient crosslinking of the GAG molecules and to ensure a sufficient number of crosslinks to obtain a durable hydrogel with desired properties (e.g., elastic modulus G′≤200 Pa). In some embodiments, during the crosslinking, the molar ratio of the crosslinker to the GAG (per GAG disaccharide) is greater than or equal to 1.5 mol %, greater than or equal to 1.6 mol %, greater than or equal to 1.7 mol %, greater than or equal to 1.8 mol %, greater than or equal to 1.9 mol %, greater than or equal to 2.0 mol %, greater than or equal to 2.1 mol %, greater than or equal to 2.2 mol %, greater than or equal to 2.3 mol %, greater than or equal to 2.4 mol %, greater than or equal to 2.5 mol %, greater than or equal to 2.6 mol %, greater than or equal to 2.7 mol %, greater than or equal to 2.8 mol %, greater than or equal to 2.9 mol %, greater than or equal to 3.0 mol %, greater than or equal to 3.1 mol %, greater than or equal to 3.2 mol %, greater than or equal to 3.3 mol %, greater than or equal to 3.4 mol %, greater than or equal to 3.5 mol %, greater than or equal to 3.6 mol %, greater than or equal to 3.7 mol %, greater than or equal to 3.8 mol %, greater than or equal to 3.9 mol %, greater than or equal to 4.0 mol %, greater than or equal to 4.5 mol %, greater than or equal to 5.0 mol %, greater than or equal to 5.5 mol %, greater than or equal to 6.0 mol %, greater than or equal to 6.5 mol %, greater than or equal to 7.0 mol %, greater than or equal to 7.5 mol %, greater than or equal to 8.0 mol %, greater than or equal to 8.5 mol %, greater than or equal to 9.0 mol %, greater than or equal to 9.5 mol %, greater than or equal to 10.0 mol %, per GAG disaccharide, or any range or value therein between.

In some embodiments, during the crosslinking, the molar ratio of the crosslinker to the GAG (per GAG disaccharide) is less than or equal to 15.0 mol %, less than or equal to 14.0 mol %, less than or equal to 13.0 mol %, less than or equal to 12.0 mol %, less than or equal to 11.0 mol %, less than or equal to 10.0 mol %, less than or equal to 9.5 mol %, less than or equal to 9.0 mol %, less than or equal to 8.5 mol %, less than or equal to 8.0 mol %, less than or equal to 7.5 mol %, less than or equal to 7.0 mol %, less than or equal to 6.5 mol %, less than or equal to 6.0 mol %, less than or equal to 5.5 mol %, less than or equal to 5.0 mol %, less than or equal to 4.5 mol %, less than or equal to 4.0 mol %, less than or equal to 3.9 mol %, less than or equal to 3.8 mol %, less than or equal to 9.7 mol %, less than or equal to 3.6 mol %, less than or equal to 3.5 mol %, less than or equal to 3.4 mol %, less than or equal to 3.3 mol %, less than or equal to 3.2 mol %, less than or equal to 3.1 mol %, less than or equal to 3.0 mol %, less than or equal to 2.9 mol %, less than or equal to 2.8 mol %, less than or equal to 2.7 mol %, less than or equal to 2.6 mol %, less than or equal to 2.5 mol %, per GAG disaccharide, or any range or value therein between.

In some embodiments, during the crosslinking, the molar ratio of the crosslinker to the GAG (per GAG disaccharide) is 1.5 mol % to 15 mol % per GAG disaccharide, 1.5 mol % to 10 mol % per GAG disaccharide, 1.5 mol % to 8.0 mol % per GAG disaccharide, 1.5 mol % to 6.0 mol % per GAG disaccharide, 1.5 mol % to 4.0 mol % per GAG disaccharide, 1.5 mol % to 3.0 mol % per GAG disaccharide, 2.0 mol % to 15 mol % per GAG disaccharide, 2.0 mol % to 10 mol % per GAG disaccharide, 2.0 mol % to 8.0 mol % per GAG disaccharide, 2.0 mol % to 6.0 mol % per GAG disaccharide, 2.0 mol % to 4.0 mol % per GAG disaccharide, 2.0 mol % to 3.0 mol % per GAG disaccharide, 3.0 mol % to 15 mol % per GAG disaccharide, 3.0 mol % to 10 mol % per GAG disaccharide, 3.0 mol % to 8.0 mol % per GAG disaccharide, 3.0 mol % to 6.0 mol % per GAG disaccharide, 3.0 mol % to 4.0 mol % per GAG disaccharide, 4.0 mol % to 15 mol % per GAG disaccharide, 4.0 mol % to 10 mol % per GAG disaccharide, 4.0 mol % to 8.0 mol % per GAG disaccharide, 4.0 mol % to 6.0 mol % per GAG disaccharide, 5.0 mol % to 15 mol % per GAG disaccharide, 5.0 mol % to 10 mol % per GAG disaccharide, 5.0 mol % to 8.0 mol % per GAG disaccharide, 5.0 mol % to 6.0 mol % per GAG disaccharide, 6.0 mol % to 15 mol % per GAG disaccharide, 6.0 mol % to 10 mol % per GAG disaccharide, 6.0 mol % to 8.0 mol % per GAG disaccharide, 8.0 mol % to 15 mol % per GAG disaccharide, 8.0 mol % to 10 mol % per GAG disaccharide, or any range or value therein between. In some embodiments, during the crosslinking, the molar ratio of the crosslinker to the GAG is greater than or equal to 1.5 mol % per GAG disaccharide and less than or equal to 10 mol % per GAG disaccharide. In some embodiments, during the crosslinking, the molar ratio of the crosslinker to the GAG is greater than or equal to 1.5 mol % per GAG disaccharide and less than or equal to 8 mol % per GAG disaccharide.

In some embodiments, the GAGs are covalently crosslinked. In some embodiments, the covalently crosslinked GAG molecules consist of, or consist essentially of, carbohydrate type structures or derivatives thereof. In some embodiments, the crosslinked GAGs or hydrogels are free of, or essentially free of, synthetic non-carbohydrate structures or linkers. This can be achieved by using a GAG in its native state together with a crosslinker which comprises, consists of, or consists essentially of carbohydrate type structures or derivatives thereof. In some embodiments, functional groups of the crosslinker are covalently bound directly to carboxyl groups of the GAG (e.g., via amide bonds). In some embodiments, the GAG is HA, and the crosslinker is DATH.

### Coupling Agent

In some embodiments, the crosslinking comprises: crosslinking activated GAG molecules via activated carboxyl groups using a di- or multi-nucleophile functional crosslinker to obtain a GAG hydrogel crosslinked by amide bonds. In some embodiments, the crosslinking comprises: activating carboxyl groups on GAG molecules with a coupling agent to form activated GAG molecules; and crosslinking the activated GAG molecules via activated carboxyl groups using a di- or multi-nucleophile functional crosslinker to obtain a GAG hydrogel crosslinked by amide bonds.

In some embodiments, the coupling agent may be selected from the group consisting of triazine-based coupling reagents, carbodiimide coupling reagents, imidazolium-derived coupling reagents, Oxyma (ethyl cyanohydroxyiminoacetate), and 1-cyano-2-ethoxy-2-oxoethylidenaminooxy)dimethylamino-morpholino-carbenium hexafluorophosphate (e.g., COMU®). In some embodiments, the coupling agent comprises a triazine-based coupling agent. In some embodiments, the triazine-based coupling agent is selected from the group consisting of 4-(4,6-dimethoxy-1,3,5-triazin-2-yl)-4-methylmorpholinium chloride (DMTMM) and 2-chloro-4,6-dimethoxy-1,3,5-triazine (CDMT). In some embodiments, the coupling agent is DMTMM.

In some embodiments, the coupling agent is a carbodiimide coupling reagent (e.g., N-(3-dimethylaminopropyl)-N′-ethylcarbodiimide (EDC) combined with N-hydroxysuccinimide (NHS)).

In some embodiments, activating carboxyl groups on GAG molecules and crosslinking the activated GAG molecules via activated carboxyl groups are carried out in a single reaction step. In some embodiments, the activation step and the crosslinking step occur simultaneously. In some embodiments, the activation step occurs prior to and separately from the crosslinking step.

### Crosslinking pH

In some embodiments, the crosslinking is performed at a pH of 5 to 9, 5 to 8, 5 to 7, 5 to 6, 6 to 9, 6 to 8, 6 to 7, 7 to 9, 7 to 8, or 8 to 9, or any range or value therein between. In some embodiments, the crosslinking is performed at a pH of about 5, about 5.5, about 6, about 6.5, about 7, about 7.5, about 8, about 8.5, or about 9.

### Hydrogel Properties

### GAG Concentration

In some embodiments, the concentration of GAG in the hydrogel is greater than or equal to about 5 mg/mL, greater than or equal to about 10 mg/mL, greater than or equal to about 15 mg/mL, greater than or equal to about 20 mg/mL, greater than or equal to about 25 mg/mL, greater than or equal to about 30 mg/mL, greater than or equal to about 40 mg/mL, greater than or equal to about 45 mg/mL, greater than or equal to about 50 mg/mL, or any range or value therein between. In some embodiments, the concentration of GAG in the hydrogel is less than or equal to about 50 mg/mL, less than or equal to about 45 mg/mL, less than or equal to about 40 mg/mL, less than or equal to about 35 mg/mL, less than or equal to about 30 mg/mL, less than or equal to about 25 mg/mL, less than or equal to about 20 mg/mL, less than or equal to about 15 mg/mL, less than or equal to about 10 mg/mL, or any range or value therein between.

In some embodiments, the concentration of GAG in the hydrogel is 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 15 mg/mL, 10 mg/mL to 50 mg/mL, 10 mg/mL to 45 mg/mL, 10 mg/mL to 40 mg/mL, 10 mg/mL to 35 mg/mL, 10 mg/mL to 30 mg/mL, 10 mg/mL to 25 mg/mL, 10 mg/mL to 20 mg/mL, 15 mg/mL to 50 mg/mL, 15 mg/mL to 45 mg/mL, 15 mg/mL to 40 mg/mL, 15 mg/mL to 35 mg/mL, 15 mg/mL to 30 mg/mL, 15 mg/mL to 25 mg/mL, 15 mg/mL to 20 mg/mL, 20 mg/mL to 50 mg/mL, 20 mg/mL to 45 mg/mL, 20 mg/mL to 40 mg/mL, 20 mg/mL to 35 mg/mL, 20 mg/mL to 30 mg/mL, 20 mg/mL to 25 mg/mL, 25 mg/mL to 50 mg/mL, 25 mg/mL to 45 mg/mL, 25 mg/mL to 40 mg/mL, 25 mg/mL to 35 mg/mL, 25 mg/mL to 30 mg/mL, 30 mg/mL to 50 mg/mL, 30 mg/mL to 45 mg/mL, 30 mg/mL to 40 mg/mL, 30 mg/mL to 35 mg/mL, 35 mg/mL to 50 mg/mL, 35 mg/mL to 45 mg/mL, 35 mg/mL to 40 mg/mL, 40 mg/mL to 50 mg/mL, or any range or value therein.

### C_{final}

In some embodiments, a hydrogel product according to the present disclosure is formulated to create a suitable GAG concentration for dermatological use, dental use, medical use, or reconstructive surgical use.

In some embodiments, the suitable GAG concentration (C_{final}) of the hydrogel product is about 5 to about 50 mg/mL, about 5 to about 45 mg/mL, about 5 to about 40 mg/mL, about 5 to about 35 mg/mL, about 5 to about 30 mg/mL, about 5 to about 25 mg/mL, about 5 to about 20 mg/mL, about 5 to about 15 mg/mL, about 5 to about 10 mg/mL, about 10 to about 50 mg/mL, about 10 to about 45 mg/mL, about 10 to about 40 mg/mL, about 10 to about 35 mg/mL, about 10 to about 30 mg/mL, about 10 to about 25 mg/mL, about 10 to about 20 mg/mL, about 10 to about 15 mg/mL, about 15 to about 40 mg/mL, about 15 to about 40 mg/mL, about 15 to about 35 mg/mL, about 15 to about 30 mg/mL, about 15 to about 25 mg/mL, about 15 to about 20 mg/mL, about 20 to about 50 mg/mL, about 20 to about 45 mg/mL, about 20 to about 40 mg/mL, about 20 to about 35 mg/mL, about 20 to about 30 mg/mL, about 20 to about 25 mg/mL, about 25 to about 50 mg/mL, about 25 to about 45 mg/mL, about 25 to about 40 mg/mL, about 25 to about 35 mg/mL, about 25 to about 30 mg/mL, about 30 to about 50 mg/mL, about 30 to about 45 mg/mL, about 30 to about 40 mg/mL, about 30 to about 35 mg/mL, about 35 to about 50 mg/mL, about 35 to about 45 mg/mL, about 35 to about 40 mg/mL, about 40 to about 50 mg/mL, or about 40 to about 45 mg/mL.

In some embodiments, the hydrogel is not subjected to a post-crosslinking degradation of the glycosaminoglycan. In some embodiments, the hydrogel is subject to ambient degradation post-crosslinking; however, the hydrogel does not exhibit a Cₘᵢₙ value below that of C_{final}/2. In some embodiments, the hydrogel exhibits a Cₘᵢₙ value greater than C_{final}/2 of the hydrogel.

In some embodiments, the hydrogel is homogeneous (i.e., not phase separated). In some embodiments, the hydrogel is formulated to a suitable concentration for dermatological use (such as 10-45 mg/mL), but retains the capacity to swell in the presence of excess saline.

In some embodiments, the method of producing a hydrogel does not result in phase separation of the hydrogel. In some embodiments, a hydrogel produced or derived from the methods disclosed herein is not phase separated. In some embodiments, the method of diluting a hydrogel after heat sterilization does not result in phase separation of the hydrogel.

### Hydrogel Product Concentration

In some embodiments, the hydrogel product is diluted in a PBS buffer for storage, transport, or use. In some embodiments, the hydrogel is diluted in about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM phosphate (PBS) buffer. In some embodiments, the hydrogel is diluted to between about 1 mM to about 20 mM, about 1 mM to about 15 mM, about 1 mM to about 10 mM, about 1 mM to about 5 mM, about 5 mM to about 20 mM, about 5 mM to about 15 mM, about 5 mM to about 10 mM, about 10 mM to about 20 mM, about 10 mM to about 15 mM, or about 15 mM to about 20 mM phosphate (PBS) buffer.

In some embodiments, the hydrogel product is diluted in a solution at a pH of about 6.0, about 6.2, about 6.4, about 6.6, about 6.8, about 7.0, about 7.2, about 7.4, about 7.6, about 7.8, or about 8.0. In some embodiments, the hydrogel is diluted in a solution at a pH of 6.0, 6.2, 6.4, 6.6, 6.8, 7.0, 7.2, 7.4, 7.6, 7.8, or 8.0. In some embodiments, the hydrogel is diluted in a solution at a pH of between 6.0 to 8.0, between 6.0 to 7.0, between 7.0 and 8.0, between 6 and 7.5, between 7.0 and 7.5, or between 6.5 and 7.5.

### Initial GelC

In some embodiments, a high proportion of the GAG molecules is bound in the hydrogel. In some embodiments, the GAG hydrogel has an initial GelC (GelCi) of at least about 65%, at least about 70%, at least about 75%, at least about 80%, at least about 85%, at least about 90%, at least about 95%, at least about 96%, at least about 97%, at least about 98%, at least about 99%, or greater. In some embodiments, the initial GelC is measured immediately after the hydrogel is prepared.

In some embodiments, the GAG hydrogel has an initial GelC (GelCi) of no greater than 99.9%, no greater than 99.5%, no greater than 99%, no greater than 98.5%, no greater than 98%, no greater than 97.5%, no greater than 97%, no greater than 96.5%, no greater than 96%, no greater than 95.5%, no greater than 95%, no greater than 94.5%, no greater than 94%, no greater than 93.5%, no greater than 93%, no greater than 92.5%, no greater than 92%, no greater than 91.5%, no greater than 91%, no greater than 90.5%, no greater than 90, or any range or value therein between.

In some embodiments, the GAG hydrogel has an initial GelC (GelCi) of 65% to 100%, 65% to 99.9%, 65% to 99.5%, 65% to 99%, 65% to 98%, 65% to 97%, 65% to 96%, 65% to 95%, 65% to 94%, 65% to 93%, 65% to 92%, 65% to 91%, 65% to 90%, 65% to 85%, 65% to 80%, 65% to 100%, 65% to 99.9%, 65% to 99.5%, 65% to 99%, 65% to 98%, 65% to 97%, 65% to 96%, 65% to 95%, 65% to 94%, 65% to 93%, 65% to 92%, 65% to 91%, 65% to 90%, 65% to 85%, 65% to 80%, 70% to 100%, 70% to 99.9%, 70% to 99.5%, 70% to 99%, 70% to 98%, 70% to 97%, 70% to 96%, 70% to 95%, 70% to 94%, 70% to 93%, 70% to 92%, 70% to 91%, 70% to 90%, 70% to 85%, 70% to 80%, 75% to 100%, 75% to 99.9%, 75% to 99.5%, 75% to 99%, 75% to 98%, 75% to 97%, 75% to 96%, 75% to 95%, 75% to 94%, 75% to 93%, 75% to 92%, 75% to 91%, 75% to 90%, 75% to 85%, 75% to 80%, 65% to 100%, 80% to 99.9%, 80% to 99.5%, 80% to 99%, 80% to 98%, 80% to 97%, 80% to 96%, 80% to 95%, 80% to 94%, 80% to 93%, 80% to 92%, 80% to 91%, 80% to 90%, 80% to 85%, 85% to 100%, 85% to 99.9%, 85% to 99.5%, 85% to 99%, 85% to 98%, 85% to 97%, 85% to 96%, 85% to 95%, 85% to 94%, 85% to 93%, 85% to 92%, 85% to 91%, 85% to 90%, 90% to 100%, 90% to 99.9%, 90% to 99.5%, 90% to 99%, 90% to 98%, 90% to 97%, 90% to 96%, 90% to 95%, or any range or value therein.

### Elastic Modulus

The GAG hydrogel may have any suitable firmness (elastic modulus, G′) for its desired application (e.g., as an injectable dermal filler). In some embodiments, the GAG hydrogel has an elastic modulus of less than or equal to 300 Pa, less than or equal to 290 Pa, less than or equal to 280 Pa, less than or equal to 270 Pa, less than or equal to 260 Pa, less than or equal to 250 Pa, less than or equal to 240 Pa, less than or equal to 230 Pa, less than or equal to 220 Pa, less than or equal to 210 Pa, less than or equal to 200 Pa, less than or equal to 190 Pa, less than or equal to 180 Pa, less than or equal to 175 Pa, less than or equal to 170 Pa, less than or equal to 165 Pa, less than or equal to 160 Pa, less than or equal to 155 Pa, less than or equal to 150 Pa, less than or equal to 145 Pa, less than or equal to 140 Pa, less than or equal to 135 Pa, less than or equal to 130 Pa, less than or equal to 125 Pa, less than or equal to 120 Pa, less than or equal to 115 Pa, less than or equal to 110 Pa, less than or equal to 100 Pa, less than or equal to 95 Pa, less than or equal to 90 Pa, less than or equal to 85 Pa, less than or equal to 80 Pa, less than or equal to 75 Pa, less than or equal to 70 Pa, less than or equal to 65 Pa, less than or equal to 60 Pa, less than or equal to 55 Pa, less than or equal to 50 Pa, less than or equal to 45 Pa, less than or equal to 40 Pa, less than or equal to 35 Pa, less than or equal to 30 Pa, less than or equal to 25 Pa, or any range or value therein between.

In some embodiments, the GAG hydrogel has an elastic modulus of greater than or equal to 0.1 Pa, greater than or equal to 0.2 Pa, greater than or equal to 0.3 Pa, greater than or equal to 0.4 Pa, greater than or equal to 0.5 Pa, greater than or equal to 1.0 Pa, greater than or equal to 1.5 Pa, greater than or equal to 2.0 Pa, greater than or equal to 2.5 Pa, greater than or equal to 3.0 Pa, greater than or equal to 3.5 Pa, greater than or equal to 4.0 Pa, greater than or equal to 4.5 Pa, greater than or equal to 5 Pa, greater than or equal to 6 Pa, greater than or equal to 7 Pa, greater than or equal to 8 Pa, greater than or equal to 9 Pa, greater than or equal to 10 Pa, greater than or equal to 15 Pa, greater than or equal to 20 Pa, greater than or equal to 25 Pa, greater than or equal to 30 Pa, greater than or equal to 35 Pa, greater than or equal to 40 Pa, greater than or equal to 45 Pa, greater than or equal to 50 Pa, greater than or equal to 55 Pa, greater than or equal to 60 Pa, greater than or equal to 65 Pa, greater than or equal to 70 Pa, greater than or equal to 75 Pa, greater than or equal to 80 Pa, greater than or equal to 85 Pa, greater than or equal to 90 Pa, greater than or equal to 95 Pa, greater than or equal to 100 Pa, greater than or equal to 110 Pa, greater than or equal to 120 Pa, or any range or value therein between.

In some embodiments, the GAG hydrogel has an elastic modulus of 0.1 Pa to 300 Pa, 0.1 Pa to 250 Pa, 0.1 Pa to 240 Pa, 0.1 Pa to 230 Pa, 0.1 Pa to 220 Pa, 0.1 Pa to 210 Pa, 0.1 Pa to 200 Pa, 0.1 Pa to 190 Pa, 0.1 Pa to 180 Pa, 0.1 Pa to 170 Pa, 0.1 Pa to 160 Pa, 0.1 Pa to 150 Pa, 0.1 Pa to 140 Pa, 0.1 Pa to 130 Pa, 0.1 Pa to 120 Pa, 0.1 Pa to 110 Pa, 0.1 Pa to 100 Pa, 1 Pa to 300 Pa, 1 Pa to 250 Pa, 1 Pa to 240 Pa, 1 Pa to 230 Pa, 1 Pa to 220 Pa, 1 Pa to 210 Pa, 1 Pa to 200 Pa, 1 Pa to 190 Pa, 1 Pa to 180 Pa, 1 Pa to 170 Pa, 1 Pa to 160 Pa, 1 Pa to 150 Pa, 1 Pa to 140 Pa, 1 Pa to 130 Pa, 1 Pa to 120 Pa, 1 Pa to 110 Pa, 1 Pa to 100 Pa, 5 Pa to 300 Pa, 5 Pa to 250 Pa, 5 Pa to 240 Pa, 5 Pa to 230 Pa, 5 Pa to 220 Pa, 5 Pa to 210 Pa, 5 Pa to 200 Pa, 5 Pa to 190 Pa, 5 Pa to 180 Pa, 5 Pa to 170 Pa, 5 Pa to 160 Pa, 5 Pa to 150 Pa, 5 Pa to 140 Pa, 5 Pa to 130 Pa, 5 Pa to 120 Pa, 5 Pa to 110 Pa, 5 Pa to 100 Pa, 10 Pa to 300 Pa, 10 Pa to 250 Pa, 10 Pa to 240 Pa, 10 Pa to 230 Pa, 10 Pa to 220 Pa, 10 Pa to 210 Pa, 10 Pa to 200 Pa, 10 Pa to 190 Pa, 10 Pa to 180 Pa, 10 Pa to 170 Pa, 10 Pa to 160 Pa, 10 Pa to 150 Pa, 10 Pa to 140 Pa, 10 Pa to 130 Pa, 10 Pa to 120 Pa, 10 Pa to 110 Pa, 10 Pa to 100 Pa, 20 Pa to 300 Pa, 20 Pa to 250 Pa, 20 Pa to 240 Pa, 20 Pa to 230 Pa, 20 Pa to 210 Pa, 20 Pa to 220 Pa, 20 Pa to 200 Pa, 20 Pa to 190 Pa, 20 Pa to 180 Pa, 20 Pa to 170 Pa, 20 Pa to 160 Pa, 20 Pa to 150 Pa, 20 Pa to 140 Pa, 20 Pa to 130 Pa, 20 Pa to 120 Pa, 20 Pa to 110 Pa, 20 Pa to 100 Pa, 50 Pa to 300 Pa, 50 Pa to 250 Pa, 50 Pa to 240 Pa, 50 Pa to 230 Pa, 50 Pa to 220 Pa, 50 Pa to 210 Pa, 50 Pa to 200 Pa, 50 Pa to 190 Pa, 50 Pa to 180 Pa, 50 Pa to 170 Pa, 50 Pa to 160 Pa, 50 Pa to 150 Pa, 50 Pa to 140 Pa, 50 Pa to 130 Pa, 50 Pa to 120 Pa, 50 Pa to 110 Pa, 50 Pa to 100 Pa, 80 Pa to 300 Pa, 80 Pa to 250 Pa, 80 Pa to 240 Pa, 80 Pa to 230 Pa, 80 Pa to 220 Pa, 80 Pa to 210 Pa, 80 Pa to 200 Pa, 80 Pa to 190 Pa, 80 Pa to 180 Pa, 80 Pa to 170 Pa, 80 Pa to 160 Pa, 80 Pa to 150 Pa, 80 Pa to 140 Pa, 80 Pa to 130 Pa, 80 Pa to 120 Pa, 80 Pa to 110 Pa, 80 Pa to 100 Pa, 100 Pa to 300 Pa, 100 Pa to 250 Pa, 100 Pa to 240 Pa, 100 Pa to 230 Pa, 100 Pa to 220 Pa, 100 Pa to 210 Pa, 100 Pa to 200 Pa, 100 Pa to 190 Pa, 100 Pa to 180 Pa, 100 Pa to 170 Pa, 100 Pa to 160 Pa, 100 Pa to 150 Pa, 100 Pa to 140 Pa, 100 Pa to 130 Pa, 100 Pa to 120 Pa, or any range or value therein.

### Gel Thermostability

GAG hydrogels prepared according to the present disclosure have surprisingly high thermal stability when compared to conventional gels of the same type (e.g., “soft” hydrogels having G′≤200 Pa). The thermal stability may be measured by determining the normalized GelC of the hydrogel (“GelC_{N}”), which is the GelC determined at a given timepoint (“GelCₜ”) divided by the initial GelC (“GelCᵢ”).

In some embodiments, the normalized GelC (GelC_{N}) of the hydrogel after 12 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater. In some embodiments, the normalized GelC (GelC_{N}) of the hydrogel after 20 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater. In some embodiments, the normalized GelC (GelC_{N}) of the hydrogel after 24 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater. In some embodiments, the normalized GelC (GelC_{N}) of the hydrogel after 36 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater. In some embodiments, the normalized GelC (GelC_{N}) of the hydrogel after 48 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater. In some embodiments, the normalized GelC (GelC_{N}) of the hydrogel after 72 hours is at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater.

In some embodiments, the normalized GelC (GelC_{N}) of the hydrogel after 8 hours, 12 hours, 16 hours, 20 hours, 24 hours, 30 hours, 36 hours, 48 hours, 60 hours, or 72 hours is 20% to 100%, 20% to 99%, 20% to 98%, 20% to 97%, 20% to 96%, 20% to 95%, 20% to 94%, 20% to 93%, 20% to 92%, 20% to 91%, 20% to 90%, 20% to 85%, 20% to 80%, 20% to 75%, 20% to 70%, 20% to 65%, 20% to 60%, 20% to 55%, 20% to 50%, 25% to 100%, 25% to 99%, 25% to 98%, 25% to 97%, 25% to 96%, 25% to 95%, 25% to 94%, 25% to 93%, 25% to 92%, 25% to 91%, 25% to 90%, 25% to 85%, 25% to 80%, 25% to 75%, 25% to 70%, 25% to 65%, 25% to 60%, 25% to 55%, 25% to 50%, 30% to 100%, 30% to 99%, 30% to 98%, 30% to 97%, 30% to 96%, 30% to 95%, 30% to 94%, 30% to 93%, 30% to 92%, 30% to 91%, 30% to 90%, 30% to 85%, 30% to 80%, 30% to 75%, 30% to 70%, 30% to 65%, 30% to 60%, 30% to 55%, 30% to 50%, 35% to 100%, 35% to 99%, 35% to 98%, 35% to 97%, 35% to 96%, 35% to 95%, 35% to 94%, 35% to 93%, 35% to 92%, 35% to 91%, 35% to 90%, 35% to 85%, 35% to 80%, 35% to 75%, 35% to 70%, 35% to 65%, 35% to 60%, 35% to 55%, 35% to 50%, 40% to 100%, 40% to 99%, 40% to 98%, 40% to 97%, 40% to 96%, 40% to 95%, 40% to 94%, 40% to 93%, 40% to 92%, 40% to 91%, 40% to 90%, 40% to 85%, 40% to 80%, 40% to 75%, 40% to 70%, 40% to 65%, 40% to 60%, 40% to 55%, 40% to 50%, 45% to 100%, 45% to 99%, 45% to 98%, 45% to 97%, 45% to 96%, 45% to 95%, 45% to 94%, 45% to 93%, 45% to 92%, 45% to 91%, 45% to 90%, 45% to 85%, 45% to 80%, 45% to 75%, 45% to 70%, 45% to 65%, 50% to 100%, 50% to 99%, 50% to 98%, 50% to 97%, 50% to 96%, 50% to 95%, 50% to 94%, 50% to 93%, 50% to 92%, 50% to 91%, 50% to 90%, 50% to 85%, 50% to 80%, 50% to 75%, 50% to 70%, 50% to 65%, 55% to 100%, 55% to 99%, 55% to 98%, 55% to 97%, 55% to 96%, 55% to 95%, 55% to 94%, 55% to 93%, 55% to 92%, 55% to 91%, 55% to 90%, 55% to 85%, 55% to 80%, 55% to 75%, 55% to 70%, 55% to 65%, 60% to 100%, 60% to 99%, 60% to 98%, 60% to 97%, 60% to 96%, 60% to 95%, 60% to 94%, 60% to 93%, 60% to 92%, 60% to 91%, 60% to 90%, 60% to 85%, 60% to 80%, 60% to 75%, 60% to 70%, 60% to 65%, 65% to 100%, 65% to 99%, 65% to 98%, 65% to 97%, 65% to 96%, 65% to 95%, 65% to 94%, 65% to 93%, 65% to 92%, 65% to 91%, 65% to 90%, 65% to 85%, 65% to 80%, 65% to 75%, 65% to 70%, 70% to 100%, 70% to 99%, 70% to 98%, 70% to 97%, 70% to 96%, 70% to 95%, 70% to 94%, 70% to 93%, 70% to 92%, 70% to 91%, 70% to 90%, 70% to 85%, 70% to 80%, 70% to 75%, 75% to 100%, 75% to 99%, 75% to 98%, 75% to 97%, 75% to 96%, 75% to 95%, 75% to 94%, 75% to 93%, 75% to 92%, 75% to 91%, 75% to 90%, 75% to 85%, 75% to 80%, 80% to 100%, 80% to 99%, 80% to 98%, 80% to 97%, 80% to 96%, 80% to 95%, 80% to 94%, 80% to 93%, 80% to 92%, 80% to 91%, 80% to 90%, 80% to 85%, 85% to 100%, 85% to 99%, 85% to 98%, 85% to 97%, 85% to 96%, 85% to 95%, 85% to 94%, 85% to 93%, 85% to 92%, 85% to 91%, 85% to 90%, 90% to 100%, 90% to 99%, 90% to 98%, 90% to 97%, 90% to 96%, 90% to 95%, 90% to 94%, 90% to 93%, 90% to 92%, 95% to 100%, 95% to 99%, 95% to 98%, 95% to 97%, 95% to 96%, or any range or value therein, at a temperature of greater than or equal to about 60° C., greater than or equal to about 65° C., greater than or equal to about 70° C., greater than or equal to about 75° C., greater than or equal to about 80° C., greater than or equal to about 85° C., greater than or equal to about 90° C., greater than or equal to about 95° C., greater than or equal to about 100° C., greater than or equal to about 105° C., greater than or equal to about 110° C., greater than or equal to about 115° C., greater than or equal to about 120° C., or any range or value therein between.

In some embodiments, the normalized GelC (GelC_{N}) is measured after holding the hydrogel at a constant temperature over 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 36 hr, 40 hr, 48 hr, 56 hr, 60 hr, 66, or 72 hr, or longer. In some embodiments, the thermal stability is determined after holding the hydrogel at a constant temperature of greater than or equal to about 60° C., greater than or equal to about 65° C., greater than or equal to about 70° C., greater than or equal to about 75° C., greater than or equal to about 80° C., greater than or equal to about 85° C., greater than or equal to about 90° C., greater than or equal to about 95° C., greater than or equal to about 100° C., greater than or equal to about 105° C., greater than or equal to about 110° C., greater than or equal to about 115° C., greater than or equal to about 120° C., or any range or value therein between.

In some embodiments, the normalized GelC (GelC_{N}) of the hydrogel after 8 hours at 90° C. is at least 85%. In some embodiments, the normalized GelC (GelC_{N}) of the hydrogel after 8 hours at 90° C. is at least 90%. In some embodiments, the normalized GelC (GelC_{N}) of the hydrogel after 8 hours at 90° C. is at least 95%.

In some embodiments, the normalized GelC (GelC_{N}) of the hydrogel after 16 hours at 90° C. is at least 75%. In some embodiments, the normalized GelC (GelC_{N}) of the hydrogel after 16 hours at 90° C. is at least 80%. In some embodiments, the normalized GelC (GelC_{N}) of the hydrogel after 16 hours at 90° C. is at least 85%. In some embodiments, the normalized GelC (GelC_{N}) of the hydrogel after 16 hours at 90° C. is at least 90%.

In some embodiments, the normalized GelC (GelC_{N}) of the hydrogel after 24 hours at 90° C. is at least 70%. In some embodiments, the normalized GelC (GelC_{N}) of the hydrogel after 24 hours at 90° C. is at least 75%. In some embodiments, the normalized GelC (GelC_{N}) of the hydrogel after 24 hours at 90° C. is at least 80%. In some embodiments, the normalized GelC (GelC_{N}) of the hydrogel after 24 hours at 90° C. is at least 85%.

In some embodiments, the normalized GelC (GelC_{N}) of the hydrogel after 30 hours at 90° C. is at least 65%. In some embodiments, the normalized GelC (GelC_{N}) of the hydrogel after 30 hours at 90° C. is at least 70%. In some embodiments, the normalized GelC (GelC_{N}) of the hydrogel after 30 hours at 90° C. is at least 75%. In some embodiments, the normalized GelC (GelC_{N}) of the hydrogel after 30 hours at 90° C. is at least 80%.

In some embodiments, the normalized GelC (GelC_{N}) of the hydrogel after 48 hours at 90° C. is at least 45%. In some embodiments, the normalized GelC (GelC_{N}) of the hydrogel after 48 hours at 90° C. is at least 50%. In some embodiments, the normalized GelC (GelC_{N}) of the hydrogel after 48 hours at 90° C. is at least 55%. In some embodiments, the normalized GelC (GelC_{N}) of the hydrogel after 48 hours at 90° C. is at least 60%. In some embodiments, the normalized GelC (GelC_{N}) of the hydrogel after 48 hours at 90° C. is at least 65%. In some embodiments, the normalized GelC (GelC_{N}) of the hydrogel after 48 hours at 90° C. is at least 70%.

The thermal stability of the hydrogel may be expressed in terms of the degradation rate, which is the change in GelC_{N} over a change in time Δt, divided by the time interval Δt. Thus, the degradation rate (ΔGelCN/Δt) may be measured in units of %/hr. In some embodiments, the hydrogel has a degradation rate, which is the change in the GelC_{N} versus time, at any of the temperatures set forth above, of less than or equal to about −3.0%/hr, less than or equal to about −2.9%/hr, less than or equal to about −2.8%/hr, less than or equal to about −2.7%/hr, less than or equal to about −2.6%/hr, less than or equal to about −2.5%/hr, less than or equal to about −2.4%/hr, less than or equal to about −2.3%/hr, less than or equal to about −2.2%/hr, less than or equal to about −2.1%/hr, less than or equal to about −2.0%/hr, less than or equal to about −1.9%/hr, less than or equal to about −1.8%/hr, less than or equal to about −1.7%/hr, less than or equal to about −1.6%/hr, less than or equal to about −1.5%/hr, less than or equal to about −1.4%/hr, less than or equal to about −1.3%/hr, less than or equal to about −1.2%/hr, less than or equal to about −1.1%/hr, less than or equal to about −1.0%/hr, less than or equal to about −0.9%/hr, less than or equal to about −0.8%/hr, less than or equal to about −0.7%/hr, less than or equal to about −0.6%/hr, less than or equal to about −0.5%/hr, less than or equal to about −0.4%/hr, less than or equal to about −0.3%/hr, less than or equal to about −0.2%/hr, less than or equal to about −0.1%/hr, or any range or value therein between. For purposes of this disclosure, when referring to a degradation rate, “less than” means closer to zero than the value stated (i.e., a degradation rate of less than-1.0%/hr could be, e.g., −0.8%/hr).

In some embodiments, the hydrogel has a degradation rate, which is the change in the GelC_{N} versus time, at any of the temperatures set forth above, of greater than or equal to about-0.01%/hr, greater than or equal to about −0.02%/hr, greater than or equal to about −0.03%/hr, greater than or equal to about −0.04%/hr, greater than or equal to about −0.05%/hr, greater than or equal to about −0.1%/hr, greater than or equal to about −0.2%/hr, greater than or equal to about-0.3%/hr, greater than or equal to about −0.4%/hr, greater than or equal to about −0.5%/hr, or any range or value therein between. For purposes of this disclosure, when referring to a degradation rate, “greater than” means further from zero than the value stated (i.e., a degradation rate of greater than-0.1%/hr could be, e.g., −0.5%/hr).

### Syringes and Kits

In some embodiments, a composition comprising a hydrogel according the present disclosure is injectable. In some embodiments, an injectable hydrogel composition is an injectable implant. In some embodiments, the present disclosure relates to an injectable implant comprising a composition comprising any hydrogel disclosed herein. In some embodiments, the injectable implant is for subdermal, intradermal, subcutaneous, intramuscular, submuscular, intragingival injection.

In some embodiments, the present disclosure relates to a pre-filled syringe comprising a composition comprising a hydrogel according to the present disclosure. In some embodiments, the disclosure relates to a pre-filled vial comprising a composition comprising a hydrogel according to the present disclosure.

In some embodiments, the present disclosure relates to a kit comprising a pre-filled syringe comprising a composition comprising a hydrogel disclosed herein. In some embodiments, a kit comprises a pre-filled vial comprising a composition comprising a hydrogel disclosed herein, a syringe, and one or more hypodermic needles. In some embodiments, the kit further comprises an antimicrobial or antiseptic composition for administering to the site of injection.

In some embodiments, the present disclosure relates to a kit for use in practicing the dermatological methods, cosmetic methods, or methods of treatment described herein. In some embodiments, kits comprise all solutions, buffers, compounds, vessels, and/or instructions sufficient for performing the methods described herein.

### Local Anesthetic

In some embodiments, a hydrogel composition further comprises a local anesthetic. In some embodiments, the hydrogel composition comprises at least one local anesthetic. In some embodiments the local anesthetic is an amide-type local anesthetic. In some embodiments, the local anesthetic is an ester-type local anesthetic.

In some embodiments, the local anesthetic is selected from the group consisting of: bupivacaine, butanilicaine, carticaine, cinchocaine (dibucaine), clibucaine, ethyl parapiperidinoacetylaminobenzoate, etidocaine, lignocaine (lidocaine), mepivacaine, oxethazaine, prilocaine, ropivacaine, tolycaine, trimecaine, vadocaine, articaine, levobupivacaine, amylocaine, cocaine, propanocaine, clormecaine, cyclomethycaine, proxymetacaine, amethocaine (tetracaine), benzocaine, butacaine, butoxycaine, butyl aminobenzoate, chloroprocaine, dimethocaine (larocaine), oxybuprocaine, piperocaine, parethoxycaine, procaine (novocaine), propoxycaine, and tricaine; or a combination thereof. In some embodiments, the local anesthetic is lidocaine.

In some embodiments, the concentration of local anesthetic in the composition is between about 1 to about 5 mg/mL. In some embodiments, the concentration of local anesthetic in the composition is between about 2 to about 4 mg/mL. In some embodiments, the concentration of local anesthetic in the composition is about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

### Additional Components

In some embodiments, a composition ccording to the present disclosure comprises a hydrogel as disclosed herein and further comprises sodium chloride. In some embodiments, the composition has a sodium chloride concentration of 0.1% w/v to 1.0% w/v (e.g., 0.1% w/v, 0.2% w/v, 0.3% w/v, 0.4% w/v, 0.5% w/v, 0.6% w/v, 0.7% w/v, 0.8% w/v, 0.9% w/v, or 1.0% w/v). In some embodiments, the composition further comprises a phosphate buffer. In some embodiments, the composition further comprises a pharmaceutically acceptable carrier. In some embodiments the composition further comprises sodium chloride, a phosphate buffer, and a pharmaceutically acceptable carrier.

In some embodiments, the composition comprises one or more density enhancing agents. In some embodiments, the density enhancing agents may be selected from sorbitol, mannitol, and fructose.

In some embodiments, the composition comprises a buffering agent. A buffering agent is a chemical compound added to a solution to allow that solution to resist changes in pH as a result of either dilution or small additions of acids or bases. Effective buffer systems employ solutions which contain large and approximately equal concentrations of a conjugate acid-base pair (or buffering agents). A buffering agent employed herein may be any such chemical compound(s) which is pharmaceutically acceptable, including but not limited to salts (conjugates acids and/or bases) of phosphates and citrates. In some embodiments, the buffering agent comprises phosphate buffered saline (PBS) or an alternative phosphate buffer.

In some embodiments, the composition is aseptic. In some embodiments, the composition is sterile. In some embodiments, the composition is sterilized via filtration sterilization, heat sterilization, or irradiation sterilization. In some embodiments, components of the composition (e.g., the hydrogel) are sterilized prior to mixing or forming the whole composition, thus resulting in a composition that comprises two or more components that were sterilized prior to preparing the composition.

### Methods of Using Hydrogels

In some embodiments, the present disclosure comprises methods of performing reparative or esthetic dermatologic treatment. In some embodiments, the reparative or esthetic dermatologic treatment comprises injecting a subject with a composition disclosed herein. In some embodiments, the injection is a subdermal, intradermal, subcutaneous, intramuscular, submuscular, or intragingival injection.

In some embodiments, methods of the present disclosure are drawn to intragingival injection to fill the gums as a result of receding gums. In some embodiments, methods are drawn to injection of the composition in one or more tissues of the oral cavity.

In some embodiments, the injection is for dermal filling, body contouring, facial contouring, and gingival filling.

In some embodiments, the injection of a composition disclosed herein is for dermal filling. In some embodiments, methods of dermal filling include injection of the composition to fill skin cracks. In some embodiments, methods of dermal filling include injection of the composition to fill fine lines in the face, neck, hands, feet, knees, and elbows. In some embodiments, methods of dermal filling include injection of the composition to fill fine wrinkles in the face, neck, hands, feet, knees, and elbows.

In some embodiments, methods of dermal filling include injection of the composition to fill scars. In some embodiments, methods of dermal filling include injection of the composition to fill depressed scars. In some embodiments, methods of dermal filling include injection of the composition to fill hypertrophic scars. In some embodiments, methods of dermal filling include injection of the composition to fill keloid scars.

In some embodiments, methods of dermal filling include injection of the composition to restore and/or correct for signs of facial fat loss (lipoatrophy) in people with human immunodeficiency virus (HIV).

In some embodiments, methods of dermal filling include injection of the composition to the backs of hands or the top of feet.

In some embodiments, methods of dermal filling include injection of the composition to strengthen weakened vocal cords.

In some embodiments, methods of dermal filling include injection of the composition to restore lost volume to a portion of the body as a result of age, illness, or injury.

In some embodiments, methods of facial contouring include injection of the composition to the face to modify the facial contour. In some embodiments, methods of facial contouring include injection of the composition to the lips to augment the size and/or shape of the lips.

In some embodiments, methods of facial contouring include injection of the composition to the face to increase facial symmetry. In some embodiments, methods of facial contouring include injection of the composition to change the shape of the face to an oval shape, round shape, square shape, triangle shape, inverted triangle shape, rectangular shape, or oblong shape. In some embodiments, methods of facial contouring include injection of the composition to increase the total width of the face. In some embodiments, methods of facial contouring include injection of the composition to increase the total length of the face.

In some embodiments, methods of facial contouring include injection of the composition to the face to increase the forehead and/or cheekbone width. In some embodiments, methods of facial contouring include injection of the composition to the face to increase the length of the jawline.

In some embodiments, methods of facial contouring include injection of the composition to the face to change the size and/or shape of the chin. In some embodiments, methods of facial contouring include injection of the composition to the face to change the size and/or shape of the forehead. In some embodiments, methods of facial contouring include injection of the composition to the face to change the size and/or shape of the cheeks. In some embodiments, methods of facial contouring include injection of the composition to the face to change the size and/or shape of the brow.

In some embodiments, methods of facial contouring include injection of the composition to the face to modify the appearance associated with retrognathia. In some embodiments, methods of facial contouring include injection of the composition to the face to modify the appearance associated with prognathism.

In some embodiments, methods of body contouring include injection of the composition to the body to modify the size and shape of various aspects of the body. In some embodiments, methods of body contouring include injection of the composition to the body to modify the size and shape of aspects of the body to increase symmetry.

In some embodiments, methods of body contouring include injection of the composition to the body to modify the size and shape of the breasts, buttocks, sacrum, groin, hips, abdomen, thorax, feet, legs, knees, popliteus, thighs, arms, hands, elbows, and/or antecubitis.

In some embodiments, methods of body contouring include injection of the composition to the body to fill a concave deformity. In some embodiments, the concave deformity is a result of age, illness, injury, or predisposition. In some embodiments, methods of body contouring include injection of the composition to the body to decrease the appearance of cellulite.

Reference will now be made in detail to some specific embodiments contemplated by the present disclosure. While various embodiments are described herein, it will be understood that it is not intended to limit the present technology to the described embodiments. On the contrary, it is intended to cover alternatives, modifications, and equivalents as may be included within the spirit and scope of the technology as defined by the appended claims.

### EXAMPLES

### Example 1. Preparation of Hydrogels

All formulations were prepared by crosslinking high molecular weight HA (3000 kDa) with diamino-functionalized trehalose (6,6′-diamino-6,6′-dideoxy-trehalose, DATH) through triazine-mediated amidation using the coupling reagent DMTMM (4-(4,6-dimethoxy-1,3,5-triazin-2-yl)-4-methylmorpholinium chloride).

DMTMM and freshly prepared stock solutions of DATH in aqueous buffer (pH 7) were mixed and added to pre-weighed HA in a reaction vessel. The HA concentration in the reaction vessel was varied between 1 wt. % and 6 wt. %. The DATH concentration was varied between 0.44 mol % to 6 mol %. The DMTMM concentration was varied between 3 mol % and 120 mol % per GAG disaccharide. The mixture was extensively mixed for 3 min, incubated at 23° C. for 24 hours, subjected to particle size reduction (PSR) using a steel mesh filter, and then was precipitated by adding 99.5% w/w ethanol. The obtained powder was further washed with ethanol and dried under vacuum overnight. Reconstitution of the dry powder in phosphate buffered saline (PBS) and 3 mg/g Lido-HCl yielded a gel product that was filled in syringes/vials and subsequently autoclaved at 125° C. for 8 minutes. The filled containers were finally placed in a 90° C. water bath, and GelC was analyzed at predetermined timepoints.

### Example 2. Stability of Crosslinked Polysaccharide Formulations

Stability of a crosslinked polysaccharide formulation can be defined as the ability of the polysaccharide formulation to maintain its initial physicochemical properties over time. Stability can be determined by measuring the decline of the gel content (GelC) over time under controlled conditions. A greater the change in GelC_{N} over time (i.e., a steeper slope of GelC_{N}) indicates a less stable formulation. Similarly, a smaller change in GelC_{N} over time (i.e., a gentler slope of GelC_{N}) indicates a more stable formulation. The change of normalized gel content (GelC_{N}) over time during 90° C. incubation was used to assess and compare stabilities of different formulations prepared according to Example 1.

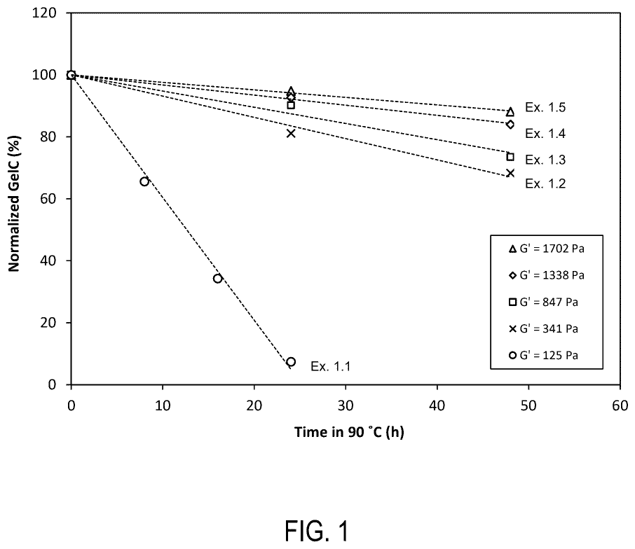

Referring to FIG. 1 and Table 1, firmer gels (having higher elastic modulus) generally exhibited greater stability compared to softer gels (having lower elastic modulus). The same has been shown for formulations prepared using higher-Mw polysaccharides compared to formulations made with lower-Mw polysaccharides, with hydrogels derived from higher-Mw polysaccharides having greater stability. See, e.g., WIPO Publication No. 2021/111303 (hereby incorporated by reference in its entirety).

TABLE 1
Reaction conditions, initial gel properties, and degradation rates
at 90° C. for formulations with different elastic moduli (firmness)

| | Reaction | | | | | |
|---|---|---|---|---|---|---|
| | Conditions | | | | | |
| | | DATH/HA | Initial Gel | | | Degrada- |
| | | (mol % per | Properties | | | tion Rate |
| Exam- | HA | GAG | HA | GelCᵢ | G′ | ΔGelC_{N}/t |
| ple | (wt. %) | disaccharide) | (mg/mL) | (%) | (Pa) | (%/hr) |
| 1.1 | 4.10 | 0.4 | 19.8 | 62 | 125 | −4.0 |
| 1.2 | 2.00 | 1.5 | 19.3 | 91 | 341 | −0.7 |
| 1.3 | 3.50 | 0.9 | 19.9 | 96 | 847 | −0.5 |
| 1.4 | 4.00 | 0.9 | 19.2 | 98 | 1338 | −0.3 |
| 1.5 | 4.00 | 1.1 | 19.6 | 99 | 1702 | −0.2 |

However, to the present inventors' surprise, the stability of softer gels (e.g., G′≤200 Pa) can be significantly increased by lowering the polysaccharide concentration and raising the amount of crosslinker used during crosslinking.

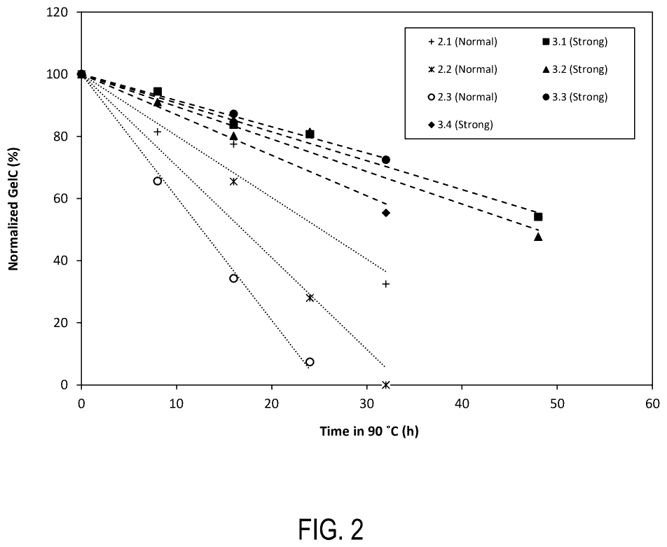

Referring now to FIG. 2 and Table 2, crosslinked hyaluronic acid “soft” gel formulations (e.g., elastic modulus (G′)≤200 Pa) were prepared using the methods discussed in Example 1. The data in Table 2 shows that formulations prepared with an HA concentration below 2 wt. % during crosslinking and a DATH/HA ratio of greater than or equal to 1.5 mol % per GAG disaccharide all have a slower degradation rate than hydrogels having similar elastic moduli, obtained using higher HA concentrations (e.g., greater than or equal to 4 wt. %) and lower DATH/HA ratios (e.g. less than 1 mol % per GAG disaccharide). Indeed, the more durable soft gel formulations exhibited a degradation rate (slope of GelC_{N} versus time) of between 0 and −1% per hour, compared to formulations prepared under normal conditions (higher HA concentration, lower DATH/HA). Results obtained from additional experiments conducted by crosslinking under reaction conditions of 2 wt. % HA combined with 2 mol % and 2.5 mol % DATH/HA, as presented in Table 3, show that using 2 wt. % HA produces firm gels with higher elastic moduli (G′) (e.g., greater than 200 Pa).

TABLE 2
Reaction conditions, initial gel properties, and degradation
rates for HA soft gel formulations at 90° C.

| | | Reaction Conditions | | | | | Degradation |
|---|---|---|---|---|---|---|---|
| | | | DATH/HA | Initial Gel Properties | | | Rate |
| | | [HA] | (mol % per GAG | HA | GelCᵢ | G′ | ΔGelC_{N}/t |
| Category | Example | (wt. %) | disaccharide) | (mg/mL) | (%) | (Pa) | (%/hr) |
| Normal | 2.1 | 4.00 | 0.3 | 20.6 | 71 | 173 | −2.0 |
| | 2.2 | 4.25 | 0.3 | 19.8 | 52 | 107 | −3.0 |
| | 2.3 (1.1) | 4.10 | 0.4 | 19.7 | 62 | 125 | −4.0 |
| Strong | 3.1 | 1.75 | 1.5 | 20.0 | 76 | 194 | −0.9 |
| | 3.2 | 1.50 | 2.0 | 19.5 | 73 | 138 | −1.0 |
| | 3.3 | 1.00 | 8.0 | 18.3 | 82 | 179 | −0.8 |
| | 3.4 | 1.00 | 6.0 | 19.0 | 78 | 129 | −1.4 |

TABLE 3
Reaction conditions and gel properties
for HA firm gel formulations

| | | Reaction Conditions | | | | |
|---|---|---|---|---|---|---|
| | | | DATH/HA | Gel Properties | | |
| Cate- | Exam- | [HA] | (mol % per GAG | HA | GelCᵢ | G′ |
| gory | ple | (wt. %) | disaccharide) | (mg/mL) | (%) | (Pa) |
| Firm | 4.1 | 2.0 | 2.0 | 19.0 | 94 | 635 |
| | 4.2 | 2.0 | 2.5 | 20.0 | 95 | 773 |

### Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, a “control” is an alternative sample used in an experiment for comparison purpose. A control can be “positive” or “negative.” A “control sample” or “reference sample” as used herein, refers to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, the term “effective amount” refers to a quantity sufficient to achieve a desired therapeutic and/or prophylactic effect, e.g., an amount which results in the prevention of one or more outcomes, or an increase in one more outcomes.

As used herein, the terms “individual”, “patient”, or “subject” can be an individual organism, a vertebrate, a mammal, or a human. In a preferred aspect, the individual, patient, or subject is a human.

As used herein, the phrase “soft tissue” refers to tissues that connect, support, or surround other structures and organs of the body. Soft tissue includes muscles, fibrous tissues, and fat.

As used herein, the phrase “soft tissue augmentation” refers to any type of volume augmentation of soft tissues, including, but not limited to facial contouring (e.g., more pronounced cheeks, chin, or lips), correction of concave deformities (e.g., post-traumatic or HIV-associated lipoatrophy), and correction of deep age-related facial folds. Thus, soft tissue augmentation may be used for cosmetic purposes or for medical purposes, such as those following trauma or degenerative disease. Soft tissue augmentation further refers to dermal filling, body contouring, and gingival filling.

As used herein, the phrase “non-animal origin” refers to a source that excludes animals, but includes sources such as yeast, bacteria, or synthetic.

As used herein, the term “bioresorbable” refers to a degradation event or events-bioresorbable substances may dissolve, may be phagocytized, or may simply degrade over a period of time such that the substances are cleared from the body, organ, tissue, location, or cell over a period of time. The substances or degradation products thereof may be metabolized, incorporated into other molecules or compounds, or excreted. In some embodiments the a hydrogel according to the present disclosure, or a composition comprising the hydrogel, is bioresorbable. In some embodiments, the hydrogel or composition comprising the hydrogel is bioresorbed within a period of about 1 year to about 3 years after administration.

As used herein, the term “aseptic” refers to something that is free or freed from pathogenic microorganisms.

As used herein, the term “sterile” refers to something that is free of living organisms, generally free of living microorganisms.

As used herein, the term “injectable” refers to the ability to inject a composition of the present disclosure through a needle.

As used herein, the terms “MW” or “Mw” refer to the mass average molecular mass.

As used herein, the term “MWₐₚₚ” refers to apparent MW, which is a simulated value for the molecular weight of GAGs in hydrogels.

As used herein, the term “SwF” refers to the swelling factor analysis in saline, which is the volume of saline for a 1 gram gel that has swelled to its maximum, usually represented in mL/g.

As used herein, the terms “gel content” or “GelC” refer to the percentage of the total GAG (e.g., HA) that is bound in gel form—that is, the amount of HA in a sample that does not pass through a 0.22-μm filter. The GelC is calculated from the amount of HA that is collected in the filtrate after filtering the hydrogel through a 0.22-μm filter and is reported as the percentage of the total amount of HA in the gel sample.

As used herein, the terms “normalized gel content,” “normalized GelC,” “NormGelC,” or “GelC_{N}” refer to the GelC measured at a given timepoint (“GelCₜ), divided by the initial GelC (GelCᵢ).

$${GelC}_{N} = \frac{{GelC}_{t}}{{GelC}_{i}}$$

As used herein, “SwD” refers to the swelling degree, which is the inverted concentration of gel-form GAG in a gel that is fully swollen in 0.9% saline, i.e., the volume or mass of a fully swollen gel that can be formed per gram of dry crosslinked GAG. The SwD generally describes the maximal liquid-absorbing (0.9% saline) capability of the product. SwD is preferably expressed in g/g, mL/g, or as a dimensionless number.

$$SwD = \frac{mass ( fully swollen gel )}{mass ( gel form GAG in fully swollen gel )}$$

The SwD also may be expressed as:

$$S w D = \frac{[ G A G ] \times G e l C}{S w F}$$

As used herein, “Crosslinker Ratio” or “CrR” (e.g., CrR_{DATH}) refers to the effective crosslinking ratio, e.g., as determined by LC-SEC-MS. The effective crosslinking ratio indicates the fraction of double-linked (“crosslinked”) crosslinker residues compared to all linked crosslinkers (including crosslinkers with only one bond to the GAG). CrR may be defined as follows:

$$C r R = \frac{mol crosslinked crosslinker}{mol linked crosslinker}$$

Thus, a CrR of 1.0 indicates that all of the crosslinker has crosslinked via two amide bonds.

As used herein, “Cₘᵢₙ” is the minimum theoretical GAG concentration—the concentration of gel-form GAGs in a gel that is fully swollen in 0.9% saline, typically expressed in mg/g or mg/mL. Cₘᵢₙ may be expressed as the inverse of the swelling degree (SwD):

$${C}_{min}^{- 1} = SwD$$

As used herein, “C_{final}” is the intended concentration of the GAG in the final hydrogel product. In some embodiments, C_{final} is greater than 2×Cₘᵢₙ.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world

What is claimed is:

1. A method of preparing a hydrogel comprising crosslinked glycosaminoglycan (GAG) molecules, the method comprising:
crosslinking a GAG having a molecular weight of at least 1.5 MDa with a crosslinker to obtain a GAG hydrogel crosslinked by amide bonds, wherein:
the concentration of GAG is less than 2 wt. % during the crosslinking;
the molar ratio of the crosslinker to the GAG is greater than or equal to 1.5 mol % per GAG disaccharide during the crosslinking; and
the GAG hydrogel has an elastic modulus (G′) of less than or equal to 200 Pa.

2. The method according to claim 1, wherein the GAG comprises hyaluronic acid (HA).

3. The method according to claim 1, wherein the crosslinker comprises a di- or multi-nucleophile functional crosslinker.

4. The method according to claim 1, wherein the crosslinker comprises one or more selected from the group consisting of di-, tri-, tetra-, and oligosaccharides.

5. The method according to claim 4, wherein the crosslinker comprises diaminotrehalose (DATH).

6. The method according to claim 1, wherein the GAG has a weight average molecular weight of about 2 MDa to about 10 MDa.

7. The method according to claim 1, wherein during the crosslinking the GAG is present at a concentration of greater than or equal to about 1.0 wt. % and less than 2 wt. %.

8. The method according to claim 1, wherein during the crosslinking, the molar ratio of the crosslinker to the GAG is greater than or equal to 1.5 mol % per GAG disaccharide and less than or equal to 10 mol % per GAG disaccharide.

9. The method according to claim 1, wherein the crosslinking comprises:
crosslinking activated GAG molecules via activated carboxyl groups using a di- or multi-nucleophile functional crosslinker to obtain a GAG hydrogel crosslinked by amide bonds.

10. The method according to claim 1, wherein the crosslinking comprises:
activating carboxyl groups on GAG molecules with a coupling agent to form activated GAG molecules; and
crosslinking the activated GAG molecules via activated carboxyl groups using a di- or multi-nucleophile functional crosslinker to obtain a GAG hydrogel crosslinked by amide bonds.

11. The method according to claim 10, wherein the coupling agent comprises 4-(4,6-dimethoxy-1,3,5-triazin-2-yl)-4-methylmorpholinium chloride (DMTMM) or N-(3-dimethylanninopropyl)-N′-ethylcarbodiimide (EDC) and N-hydroxysuccinimide (NHS).

12. The method according to claim 1, wherein the crosslinking is performed at a pH of 5 to 9.

13. The method according to claim 1, wherein the GAG hydrogel has an elastic modulus (G′) of less than or equal to 180 Pa.

14. The method according to claim 1, wherein the GAG hydrogel product has a degradation rate of less than or equal to 2.0% per hour at 90° C.

15. The method according to claim 1, wherein the GAG hydrogel has a GelC_{N} of greater than or equal to about 90% after 8 hr at 90° C.

16. The method according to claim 1, wherein the GAG hydrogel has a GelC_{N} of greater than or equal to about 75% after 24 hr at 90° C.

17. A GAG hydrogel comprising crosslinked glycosaminoglycan (GAG) molecules, wherein the GAG hydrogel is prepared according to the method according to claim 1.

18. A GAG hydrogel, comprising:
hyaluronic acid (HA) molecules crosslinked by diaminotrehalose (DATH) through amide bonds between the HA molecules and the DATH, wherein:
the HA has a weight average molecular weight of greater than or equal to 2 MDa;
the molar ratio of the crosslinker to the HA is greater than or equal to 1.5 mol % per GAG disaccharide; and
the GAG hydrogel has an elastic modulus (G′) of less than or equal to 200 Pa.

19. The GAG hydrogel according to claim 18, wherein:
the HA has a weight average molecular weight of greater than or equal to 3 MDa; and
the molar ratio of the crosslinker to the HA is 1.5 to 8 mol % per GAG disaccharide.

20. The GAG hydrogel according to claim 18, wherein the molar ratio of the crosslinker to the HA is 2 to 6 mol % per GAG disaccharide.
